(19) United States
(12) Patent Application Publication
(10) Pub. No.: US 2015/0165061 A1
(43) Pub. Date: Jun. 18, 2015

(54) INTRANASAL DELIVERY OF CELL PERMEANT THERAPEUTICS FOR THE TREATMENT OF EDEMA

(71) Applicant: THE TRUSTEES OF COLUMBIA UNIVERSITY IN THE CITY OF NEW YORK, New York, NY (US)

(72) Inventors: Carol M. Troy, Hastings-on-Hudson, NY (US); Nsikan E. Akpan, New York, NY (US)

(73) Assignee: THE TRUSTEES OF COLUMBIA UNIVERSITY IN THE CITY OF NEW YORK, New York, NY (US)

(21) Appl. No.: 14/568,710

(22) Filed: Dec. 12, 2014

Related U.S. Application Data
(63) Continuation of application No. PCT/US2013/045674, filed on Jun. 13, 2013.
(60) Provisional application No. 61/659,096, filed on Jun. 13, 2012.

(51) Int. Cl. A61K 47/48; A61K 38/16; A61K 9/00

(52) U.S. Cl. CPC A61K 47/48246; A61K 9/0043; A61K 38/16

(57) ABSTRACT

The present invention relates to compositions and methods for the inhibition of edema, including, but not limited to, edema associated with ischemic injury in the CNS. For example, in certain embodiments, the instant invention relates to methods and compositions for the inhibition of caspase-9 activity associated with the induction and/or exacerbation of edema.

### CROSS REFERENCE TO RELATED APPLICATIONS

This application is a continuation of International Application No. PCT/US2013/045674, filed on Jun. 13, 2013 which claims the benefit of U.S. Provisional Application No. 61/659,096, filed on Jun. 13, 2012, the contents of each of which are hereby incorporated by reference in their entireties.

### STATEMENT REGARDING FEDERALLY SPONSORED RESEARCH OR DEVELOPMENT

This invention was made with government support under grant numbers NSO43089 and NS035933 awarded by NIH-NINDS. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 3, 2014, is named 0700505361SeqList.txt, and is 4,479 bytes in size. The sequence listing submitted herewith, does not extend beyond the scope of the specification, and thus, does not contain new matter.

1. Introduction

The present invention relates to compositions and methods for the inhibition of edema, including, but not limited to, edema associated with ischemic injury in the central nervous system (“CNS”).

2. Background of the Invention

Stroke is the 3rd largest cause of death and the largest cause of disability in the U.S., yet there is no effective therapy for the vast majority of cases. Present therapeutic options, including pharmacological and mechanical thrombolysis, merely aim to restore blood flow in the hopes of salvaging at-risk tissue. However, these strategies do not target the underlying causes of stroke-induced injuries, including stroke-induced edema. Development of effective therapies has been hindered by lack of knowledge of the signaling pathways critical to such injuries. Thus, elucidation of the key molecular participants, as well as methods of specifically inhibiting such molecules, will be instrumental in developing new therapeutics for stroke-induced injuries.

Edema in general, and stroke-induced edema such as cerebral edema, in particular, is caused by a localized loss of vascular integrity. This loss of vascular integrity commonly takes the form of the elimination of tight junctions between the cells of the blood vessels, rather than the outright death of the endothelial cells and pericytes that make up the blood vessels. For example, in the case of cerebral edema, the elimination of tight junctions allows for the extravasation of fluid from small intracranial blood vessels.

Edema formation is a major contributor to death and disability in severe stroke. Fatality within the first 3 days following a stroke is nearly always due to edema. Edema becomes evident within 12 hours after a large stroke and continues to develop over the next few days. Rosenberg et al., Neurosurg Focus 22 (5), E4 (2007). At present, a common edema treatment is craniectomy (the physical removal of a piece of the skull). This intervention decompresses the brain, which preserves the brainstem and respiratory function. However, this therapy does not resolve the edema, which continues to destroy the brain parenchyma and contributes to long-lasting disability. Walcott et al., PLoS One 6 (12), e29193 (2011). The only medicinal therapy currently in use is the administration of mannitol, which alters blood osmolality. Other, non-medicinal, treatments include hypoventilation and hypothermia. Simard et al., Curr Treat Options Neurol 13 (2), 217-229 (2011). Unfortunately, these therapies have transient effects, and none have proven completely satisfactory. Lopez et al., Neurotherapeutics 8 (3), 414-424 (2011).

For more than a decade, the caspase family of death proteases has been implicated in cerebral ischemia and neurodegeneration. Recent evidence shows that distinct caspase pathways are activated during ischemia. For example, the caspase-9/-6 pathway is responsible for neuronal dysfunction and death after ischemia. This data suggest that inhibiting caspase-9 activity provides substantial neuroprotection following an ischemic insult. As discussed in detail herein, caspase-9 activity is not only involved in neuronal degeneration, but also plays a role in the development of cerebral edema.

In light of the foregoing, the instant application provides methods and compositions for the inhibition of edema, including, but not limited to, cerebral edema. In particular, the instant application is directed to methods and compositions for the inhibition of caspase-9 signaling activity associated with the induction and/or exacerbation of edema.

### 3. SUMMARY OF THE INVENTION

In certain embodiments, the instant invention is directed to methods of treating edema comprising administering an effective amount of a caspase-9 inhibitor to a subject in need thereof.

In certain embodiments, the instant invention is directed to methods of treating edema comprising administering, intranasally, an effective amount of a caspase-9 inhibitor to a subject in need thereof.

In certain embodiments, the instant invention is directed to methods of treating edema comprising administering, intranasally, an effective amount of a caspase-9 inhibitor to a subject in need thereof, wherein the caspase-9 inhibitor is conjugated to a cell-penetrating peptide.

In certain embodiments, the instant invention is directed to methods of treating edema associated with ischemic injury in the central nervous system comprising administering, intranasally, an effective amount of a caspase-9 inhibitor to a subject in need thereof.

In certain embodiments, the instant invention is directed to methods of treating edema associated with ischemic injury in the central nervous system comprising administering, intranasally, an effective amount of a caspase-9 inhibitor to a subject in need thereof, wherein the cell-penetrating peptide is selected from the group consisting of Penetratin1, transportan, pIS1, Tat(48-60), pVEC, MAP, and MTS.

In certain embodiments of the invention, the caspase-9 inhibitor is selected from the group consisting of: a small molecule inhibitor; a polypeptide inhibitor; and a nucleic acid inhibitor.

In certain embodiments of the invention, the caspase-9 inhibitor is XBIR3.

In certain embodiments of the invention, the caspase-9 inhibitor is XBIR3 linked to Penetratin1.

### 4. BRIEF DESCRIPTION OF THE FIGURES

FIG. 1. Caspase-9 is activated in cerebral ischemia. bVAD was infused using CED into the predicted stroke area of rats before tMCAo. Animals were harvested at 1 hpr, and bVAD-caspase complexes were isolated and analyzed by western blotting. “I”=ipsolateral (stroked) hemisphere; “C”=contralateral (nonstroked) hemisphere.

FIG. 2A-B. Pen1-XBIR3 is neuroprotective against cerebral ischemia.

FIG. 2A Intranasal Pen1-XBIR3 protects neurons. Vehicle or Pen1-XBIR3 was delivered, and rats were harvested at 12 hpr (left) and 24 hpr (right). Sections were immunostained for NeuN (green), and NeuN-positive cells were quantified. Scale bars, 50 μm. Graphs are means (with SEM) of axon counts and neuron counts at 12 hpr (left) and 24 hpr (right). FIG. 2B Intranasal Pen1-XBIR3 provides long-term protection from stroke. 2 hr tMCAo was performed on rats given either prophylactic (pre-stroke) intranasal vehicle (black squares) or prophylactic Pen1-XBIR3 (blue triangles) or therapeutic (poststroke) Pen1-XBIR3 (red circles). Rats were monitored for 21 d. Control are unstroked animals given vehicle or Pen1-XBIR3. Means (with SEM) of neurofunctional scores are shown. ANOVA is shown. Asterisks indicate stroked prophylactic vehicle versus prophylactic or therapeutic Pen1-XBIR3 (n=8 per group, p<0.05).

FIG. 3. Inhibition of caspase-9 decreases edema and infarct volume. Vehicle or Pen1-XBIR3 was delivered intranasally and rats were harvested at 24 hpr. Sections were stained with H & E. Representative sections are shown. Direct stroke volume (infarct area/ipsilateral hemisphere area) and indirect stroke volume (infarct area/contralateral hemisphere) were quantified, n=3, ANOVA p<0.05.

FIG. 4A-C. tMCAo induces caspase-9 in blood vessels. FIG. 4A Rats were subjected to tMCAo, harvested at 4 hpr. Brain sections were immunostained for caspase-9 (green) and a marker for endothelial cells, CD34 (red). FIG. 4B Sections from control animal, stained as in FIG. 4A. FIG. 4C Sections from FIG. 4A, stained for caspase-9 (green) and a marker for pericytes, SMA (red).

FIG. 5. Cleaved-caspase-3, cl-caspase-6 are not induced in blood vessels, cells are not dying. Rats were subjected to tMCAo and brains harvested at 12 hpr. Sections were imaged for caspase-9 (left), cl-caspase-6 (center), cl-caspase-3 (right) and ToPro3 was used to visualize nuclei. The nuclei of the blood vessel cells appear healthy.

FIG. 6. Inhibition of caspase-9 abrogates tMCAo induction of MMP-9. Rats were treated with Pen1-XBIR3 or vehicle prior to tMCAo. Brains were harvested at 24 hpr and lysates analyzed by western blotting. Blots show brains from 2 rats for each treatment.

FIG. 7A-B. Small blood vessels contain p75NTR. Rat brain sections were immunostained for p75NTR and markers of pericytes (SMA) and endothelial cells (CD34). FIG. 7A green=p75NTR and red=SMA. FIG. 7B green=p75NTR and red=CD34.

FIG. 8. proNGF increases after tMCAo, proBDNF does not increase. Rats were subjected to tMCAo, harvested at the indicated times and brain lysates analyzed by western blot for proNGF and proBDNF expression. Blots show brains from 2 rats per time point. ERKs are used as loading control.

FIG. 9. Intranasal Pen1-XBir3 prevents extravasation of Evan's blue albumin (EBA) induced by tMCAo. Rats were subjected to tMCAo after intranasal treatment with Pen1-XBir3 or vehicle. EBA was injected via tail vein 30 min before harvest; rats were harvested at 4 hpr. EBA in brain tissue was quantified spectrophotometrically, n=6.

FIG. 10. Illustrates the caspase-9 signaling pathway leading to edema. Briefly, induction of proNGF leads to p75NTR activation of caspase-9. Caspase-9 cleaves TIMP-1, leading to a release of TIMP-1 inhibition of MMP-9, increasing MMP-9. Both caspase-9 and MMP-9 cleave vascular substrates that maintain vascular integrity; endothelial tight junctions are broached and the vessels leak.

### 6. DETAILED DESCRIPTION OF THE INVENTION

### 5.1 Methods of Inhibiting Edema

The present invention relates to compositions and methods for the inhibition of edema, including, but not limited to, edema associated with ischemic injury in the CNS. For example, in certain embodiments, the instant invention relates to methods and compositions for the inhibition of caspase-9 signaling activity associated with the induction and/or exacerbation of edema.

As used herein, “edema” relates to the swelling in any organ or tissue caused by increased interstitial fluid. In certain instances, increased secretion of fluid into or impaired removal of fluid from the interstitium may upset interstitial fluid homeostasis, thereby causing edema. Edema can be caused by: (1) increased hydrostatic pressure; (2) reduced oncotic pressure (osmotic pressure due to plasma proteins); (3) lymphatic obstruction; (4) destruction or removal of lymph vessels (e.g., by radiotherapy or surgery; (5) sodium retention; and/or (6) inflammation (e.g. from infection).

### 5.1.1. Stroke-Induced Edema

Stroke-induced edema is life-threatening due to the fact that swelling of the brain leads to herniation, compression of the brainstem, and ultimately death. In cerebral ischemia, edema has been suggested to occur in two stages. Yang et al., Stroke 42 (11), 3323-3328 (2011). In the first stage, a transient opening of the blood brain barrier (BBB) occurs at the time of reperfusion leading to a fluid influx. In the second stage, which occurs after reperfusion, the BBB becomes disrupted and edema becomes evident structurally 12-24 hours post reperfusion. Thus, edema can be described as “cytotoxic,” via swelling and bursting of cells, or “vasogenic,” where the BBB is disrupted.

The BBB, or neurovascular unit, is the interface between the peripheral circulatory system and the brain parenchyma. Ribe et al., Biochem J 415 (2), 165-182 (2008). It is composed of neurons, astrocytes and blood vessels. A key aspect of this barrier between the peripheral circulation and the brain is the tight junction of the endothelial cells of the small capillaries. The second stage of cerebral edema, where the BBB becomes disrupted, occurs when a loss of vascular integrity of small capillaries allows extravasation of fluid into the adjacent tissue and vasogenic edema results.

Small blood vessels, such as those present at the BBB, are composed of an inner layer of endothelial cells, which are connected by tight junctions and surrounded by a layer of pericytes in a loose extracellular matrix. Pericytes are the contractile cells of the small blood vessels. Diaz-Flores et al., Histology and Histopathology 24 (7), 909-969 (2009). Tight junction proteins responsible for maintaining the vascular integrity of such capillaries include transmembrane proteins (occludin, claudins-3, -5, -12, junction adhesion molecule-1 (JAM-1)) and cytoplasmic proteins (zona occludens-1 and 2 (ZO-1, ZO-2), cingulin, AF-6 and 7H6) linked to the cytoskeleton. Candelario-Jalil et al., Brain Edema in Neurological Diseases in Neurochemical Mechanisms in Disease, edited by J. P. Blass (Springer, New York, 2011), Vol. 1, pp. 125-168. Aquaporin-4, expressed on perivascular astrocyte end feet and on endothelial cells, is also a major regulator of fluid entry/exit in the brain. Yang et al., Stroke 42 (11), 3323-3328 (2011). In addition, during ischemia, there is an increase in several matrix metalloproteinases (MMPs), particularly MMP-9. This elevation has been correlated with a decrease in claudin-5 and ZO-1 as well as the development of edema. Yang et al., Methods in molecular biology 762, 333-345 (2011), with MMP-9-null animals exhibit less edema after ischemia. Lee et al., The Journal of Neuroscience 24 (3), 671-678 (2004). As outlined in Section 5.1.3, below, and without being bound by any particular theory of therapeutic action, the methods and compositions of the instant invention target the regulation of such tight junctions in order to inhibit edema associated with stroke.

### 5.1.2. Other Edemas

As outlined herein, the types of edema that can be inhibited using the methods and/or compositions of the instant invention not only include stroke-induced edema, such as cerebral edema, but also additional types of edema, such as, but not limited to: edema associated with traumatic brain injury; pulmonary edema; angioedema; cardiac edema; macular edema; and peripheral edema.

### 5.1.3. Therapeutic Methods

In certain embodiments, the instant invention is directed to methods of ameliorating the impact of and/or inhibiting the induction and/or exacerbation of edema. For example, in certain embodiments, the instant invention is directed to methods of administering an effective amount of a caspase-9 signalling pathway inhibitor, or conjugate thereof, in order to inhibit edema. In certain embodiments, the edema treated in this manner is: edema associated with ischemic injury; edema associated with traumatic brain injury; pulmonary edema; angioedema; cardiac edema; macular edema; or peripheral edema.

In certain embodiments, the instant invention is directed to methods of ameliorating the impact of CNS ischemic injury-associated edema. For example, in certain embodiments, the instant invention is directed to methods of administering an effective amount of a caspase-9 signalling pathway inhibitor, or conjugate thereof, in order to inhibit cerebral edema.

In certain embodiments, the methods of the instant invention are directed to the intranasal administration of a caspase-9 signalling pathway inhibitor, or conjugate thereof, in order to inhibit edema. In certain embodiments, the edema treated in this manner is: edema associated with ischemic injury; edema associated with traumatic brain injury; pulmonary edema; angioedema; cardiac edema; macular edema; or peripheral edema.

In certain non-limiting embodiments of the instant invention, the caspase-9 signalling pathway inhibitor, or conjugate thereof, is administered during a treatment window that corresponds to the particular edema being treated.

In certain non-limiting embodiments of the instant invention relating to ischemic injury-associated edema, the caspase-9 signalling pathway inhibitor, or conjugate thereof, is administered during a treatment window that begins at the onset of ischemia and extends over the next 48 hours, where treatment is can be administered within about 24 hours or within about 12 hours of the ischemic event. In additional non-limiting embodiments, the methods of the invention may be used to treat a patient who has experienced a sudden onset of a neurological deficit that would be consistent with a diagnosis of cerebral infarction or transient ischemic attack; for example, such neurologic deficit may be an impairment of speech, sensation, or motor function, in order to inhibit the induction or exacerbation of ischemic injury-associated edema.

The treatment, when used to treat/ameliorate the effects of edema, may be administered as a single dose or multiple doses. For example, but not by way of limitation, in the context of ischemic injury-associated edema, where multiple doses are administered, they may be administered at intervals of 6 times per 24 hours or 4 times per 24 hours or 3 times per 24 hours or 2 times per 24 hours. In certain embodiments, the initial dose may be greater than subsequent doses or all doses may be the same.

In certain specific, non-limiting examples of the instant invention, Pen1-XBIR3, discussed in detail in section 5.2.3, below, is employed to treat edema.

In such examples, the Pen1-XBIR3 conjugate is administered to a patient suffering from an ischemic injury either as a single dose or in multiple doses. Where multiple doses are administered, they may be administered at intervals of 6 times per 24 hours or 4 times per 24 hours or 3 times per 24 hours or 2 times per 24 hours. The initial dose may be greater than subsequent doses or all doses may be the same. The concentration of the Pen1-XBIR3 composition administered is, in certain embodiments: 0.01 μM to 1000 μM; 1 μM to 500 μM; or 10 μM to 100 μM). The Pen1-XBIR3 composition is delivered nasally by administering, in certain embodiments, drops of 0.1 μl to 1000 μl; 1.0 μl to 500 μl; or 10 μl to 100 μl to alternating nares every 30 seconds to five minutes; every one minute to every four minutes; or every two minutes for 10 to 60 minutes; every 15 to 30 minutes; or every 20 minutes. In certain embodiments, a specific human equivalent dosage can be calculated from animal studies via body surface area comparisons, as outlined in Reagan-Shaw et al., FASEB J., 22; 659-661 (2007).

In certain specific, non-limiting examples of the instant invention, Pen1-XBIR3 is employed to treat neurodegenerative disease. In such examples, the Pen1-XBIR3 conjugate is administered to a patient suffering from a neurodegenerative disease either as a single dose or in multiple doses. Where multiple doses are administered, they may be administered at intervals of 6 times per 24 hours or 4 times per 24 hours or 3 times per 24 hours or 2 times per 24 hours. The initial dose may be greater than subsequent doses or all doses may be the same. The concentration of the Pen1-XBIR3 composition administered is, in certain embodiments: 0.01 μM to 1000 μM; 1 μM to 500 μM; or 10 μM to 100 μM). The Pen1-XBIR3 composition is delivered nasally by administering, in certain embodiments, drops of 0.1 μl to 1000 μl; 1.0 μl to 500 μl; or 10 μl to 100 μl to alternating nares every 30 seconds to five minutes; every one minute to every four minutes; or every two minutes for 10 to 60 minutes; every 15 to 30 minutes; or every 20 minutes. In certain embodiments, a specific human equivalent dosage can be calculated from animal studies via body surface area comparisons, as outlined in Reagan-Shaw et al., FASEB J., 22; 659-661 (2007).

In certain embodiments of the instant invention, the caspase-9 signaling pathway inhibitor, either alone or in the context of a membrane-permeable conjugate is administered in conjunction with one or more additional therapeutics. In certain of such embodiments the additional therapeutics include, but are not limited to, anticoagulant agents, such as tPA or heparin, free radical scavengers, anti-glutamate agents, etc. (see, for example, Zaleska et al., 2009, Neuropharmacol. 56(2):329-341). In certain embodiments the method involves the administration of one or more additional caspase-9 signaling pathway inhibitors either alone or in the context of a membrane-permeable conjugate.

### 5.2 Compositions for the Inhibition of Edema

### 5.2.1 Caspase-9 Inhibitors

In certain embodiments, the instant invention relates to inhibitors of caspase-9. In certain embodiments, the caspase-9 inhibitors of the instant invention are selected from the group consisting of small molecule inhibitors, peptide/protein inhibitors, and nucleic acid inhibitors. Such inhibitors can exert their function by inhibiting either the expression or activity of caspase-9.

In certain embodiments, the caspase-9 inhibitors of the instant invention include small molecule inhibitors of caspase-9. In certain embodiments the small molecule inhibitors of caspase-9 include, but are not limited to, isatin sulfonamides (Lee, et al., J Biol Chem 275:16007-16014 (2000); Nuttall, et al., Drug Discov Today 6:85-91 (2001)), anilinoquinazolines (Scott, et al., JPET 304 (1) 433-440 (2003), and one or more small molecule caspase-9 inhibitor disclosed in U.S. Pat. No. 6,878,743.

In certain embodiments, the caspase-9 inhibitors of the instant invention are peptide inhibitors of caspase-9. In certain embodiments the peptide inhibitors of caspase-9 include, but are not limited to EG Z-VEID-FMK (“VEID” disclosed as SEQ ID NO: 1) (WO 2006056487); Z-VAD-FMK, CrmA, and Z-VAD-(2,6-dichlorobenzoyloxopentanoic acid) (Garcia-Calvo, et al., J. Biol. Chem., 273, 32608-32613 (1998)).

In certain embodiments, the caspase-9 inhibitors include, but are not limited to the class of protein inhibitors identified as Inhibitors of Apoptosis (“IAPs”). IAPB generally contain one to three BIR (baculovirus IAP repeats) domains, each consisting of approximately 70 amino acid residues. In addition, certain IAPB also have a RING finger domain, defined by seven cysteines and one histidine (e.g. C3HC4) that can coordinate two zinc atoms. Exemplary mammalian IAPB, such as, but not limited to c-IAP1 (Accession No. Q13490.2), cIAP2 (Accession No. Q13489.2), and XIAP (Accession No. P98170.2), each of which have three BIRs in the N-terminal portion of the molecule and a RING finger at the C-terminus. In contrast, NAIP (Accession No. Q13075.3), another exemplary mammalian IAP, contains three BIRs without RING, and survivin (Accession No. 015392.2) and BRUCE (Accession No. Q9H8B7), which are two additional exemplary IAPB, each has just one BIR.

Polypeptide caspase-9 inhibitors include those amino acid sequences that retain certain structural and functional features of the identified caspase-9 inhibitor polypeptides, yet differ from the identified inhibitors' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions. As used herein, a “conservative amino acid substitution” is intended to include a substitution in which the amino acid residue is replaced with an amino acid residue having a similar side chain. Families of amino acid residues having similar side chains have been defined in the art, including: basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine); nonpolar side chains (e.g., alanine, valine, leucine, isoleucine, proline, phenylalanine, methionine, tryptophan); β-branched side chains (e.g., threonine, valine, isoleucine); and aromatic side chains (e.g., tyrosine, phenylalanine, tryptophan, histidine). Other generally preferred substitutions involve replacement of an amino acid residue with another residue having a small side chain, such as alanine or glycine. Amino acid substituted peptides can be prepared by standard techniques, such as automated chemical synthesis.

In certain embodiments, a polypeptide inhibitor of the present invention is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the original caspase-9 inhibitor, such as an IAP, and is capable of caspase-9 inhibition. As used herein, the percent homology between two amino acid sequences may be determined using standard software such as BLAST or FASTA. The effect of the amino acid substitutions on the ability of the synthesized polypeptide to inhibit caspase-9 can be tested using the methods disclosed in Examples section, below.

In certain non-limiting embodiments, the caspase-9 inhibitors of the instant invention are nucleic acid inhibitors. In certain embodiments, such nucleic acid inhibitors include, but are not limited to, inhibitors that function by inhibiting the expression of the target, such as ribozymes, antisense oligonucleotide inhibitors, and siRNA inhibitors. A “ribozyme” refers to a nucleic acid capable of cleaving a specific nucleic acid sequence. Within some embodiments, a ribozyme should be understood to refer to RNA molecules that contain anti-sense sequences for specific recognition, and an RNA-cleaving enzymatic activity, see, for example, U.S. Pat. No. 6,770,633. In contrast, “antisense oligonucleotides” generally are small oligonucleotides complementary to a part of a gene to impact expression of that gene. Gene expression can be inhibited through hybridization of an oligonucleotide to a specific gene or messenger RNA (mRNA) thereof. In some cases, a therapeutic strategy can be applied to dampen expression of one or several genes believed to initiate or to accelerate inflammation, see, for example, U.S. Pat. No. 6,822,087 and WO 2006/062716. A “small interfering RNA” or “short interfering RNA” or “siRNA” or “short hairpin RNA” or “shRNA” are forms of RNA interference (RNAi). An interfering RNA can be a double-stranded RNA or partially double-stranded RNA molecule that is complementary to a target nucleic acid sequence, for example, caspase 6 or caspase 9. Micro interfering RNA's (miRNA) also fall in this category. A double-stranded RNA molecule is formed by the complementary pairing between a first RNA portion and a second RNA portion within the molecule. The length of each portion generally is less than 30 nucleotides in length (e.g., 29, 28, 27, 26, 25, 24, 23, 22, 21, 20, 19, 18, 17, 16, 15, 14, 13, 12, 11, or 10 nucleotides). In some embodiments, the length of each portion is 19 to 25 nucleotides in length. In some siRNA molecules, the complementary first and second portions of the RNA molecule are the “stem” of a hairpin structure. The two portions can be joined by a linking sequence, which can form the “loop” in the hairpin structure. The linking sequence can vary in length. In some embodiments, the linking sequence can be 5, 6, 7, 8, 9, 10, 11, 12 or 13 nucleotides in length. Linking sequences can be used to join the first and second portions, and are known in the art. The first and second portions are complementary but may not be completely symmetrical, as the hairpin structure may contain 3′ or 5′ overhang nucleotides (e.g., a 1, 2, 3, 4, or 5 nucleotide overhang). The RNA molecules of the invention can be expressed from a vector or produced chemically or synthetically.

### 5.2.2 Other Caspase-9 Signaling Pathway Inhibitors

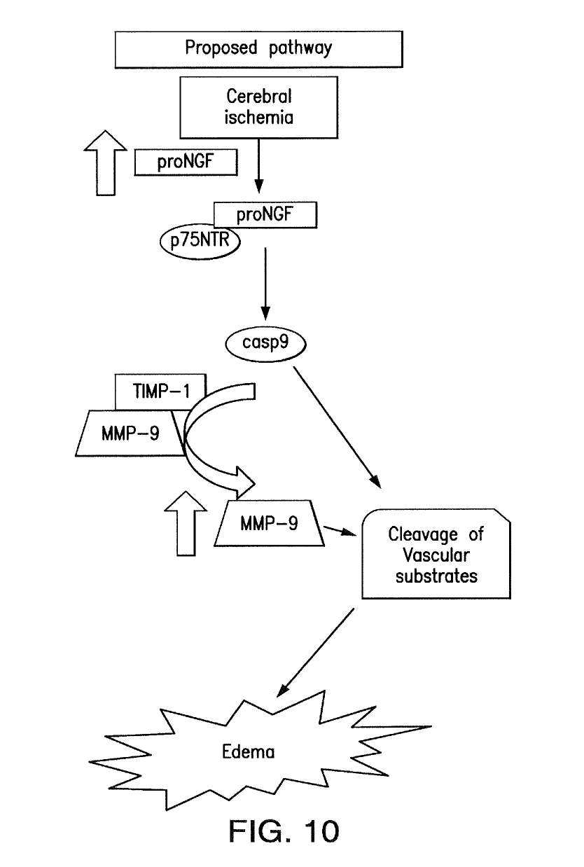

In addition to specific inhibitors of caspase-9, the instant invention relates to compositions comprising inhibitors of other members of the caspase-9 signaling pathway. For example, in cerebral ischemia, the induction of proNGF as a result of the ischemic event leads to p75NTR activation of caspase-9 (see FIG. 10). Caspase-9 cleaves TIMP-1, leading to a release of TIMP-1 inhibition of MMP-9, increasing MMP-9. Thus, in certain embodiments such inhibitors include, but are not limited to, inhibitors of proNGF, p75NTR, and/or MMP-9 activity or an inhibitor of the release of TIMP-1-mediated inhibition of MMP-9. In certain embodiments, the caspase-9 pathway inhibitors of the instant invention are selected from the group consisting of small molecule inhibitors, peptide/protein inhibitors, and nucleic acid inhibitors. Such inhibitors can exert their function by inhibiting either the expression or activity of members of the caspase-9 signaling pathway.

In certain non-limiting embodiments, the caspase-9 pathway inhibitor is a nucleic acid inhibitor of a member of the caspase-9 pathway. For example, but not by way of limitation, the caspase-9 pathway inhibitors of the instant invention which are nucleic acids include, but are not limited to, inhibitors that function by inhibiting the expression of the caspase-9 pathway target, such as ribozymes, antisense oligonucleotide inhibitors, and siRNA inhibitors. A “ribozyme” refers to a nucleic acid capable of cleaving a specific nucleic acid sequence. Within some embodiments, a ribozyme should be understood to refer to RNA molecules that contain anti-sense sequences for specific recognition, and an RNA-cleaving enzymatic activity, see, for example, U.S. Pat. No. 6,770,633. In contrast, “antisense oligonucleotides” generally are small oligonucleotides complementary to a part of a gene to impact expression of that gene. Gene expression can be inhibited through hybridization of an oligonucleotide to a specific gene or messenger RNA (mRNA) thereof. In some cases, a therapeutic strategy can be applied to dampen expression of one or several genes believed to initiate or to accelerate inflammation, see, for example, U.S. Pat. No. 6,822,087 and WO 2006/062716. A “small interfering RNA” or “short interfering RNA” or “siRNA” or “short hairpin RNA” or “shRNA” are forms of RNA interference (RNAi). An interfering RNA can be a double-stranded RNA or partially double-stranded RNA molecule that is complementary to a target nucleic acid sequence, for example, caspase 6 or caspase 9. Micro interfering RNA's (miRNA) also fall in this category. A double-stranded RNA molecule is formed by the complementary pairing between a first RNA portion and a second RNA portion within the molecule. The length of each portion generally is less than 30 nucleotides in length (e.g., 29, 28, 27, 26, 25, 24, 23, 22, 21, 20, 19, 18, 17, 16, 15, 14, 13, 12, 11, or 10 nucleotides). In some embodiments, the length of each portion is 19 to 25 nucleotides in length. In some siRNA molecules, the complementary first and second portions of the RNA molecule are the “stem” of a hairpin structure. The two portions can be joined by a linking sequence, which can form the “loop” in the hairpin structure. The linking sequence can vary in length. In some embodiments, the linking sequence can be 5, 6, 7, 8, 9, 10, 11, 12 or 13 nucleotides in length. Linking sequences can be used to join the first and second portions, and are known in the art. The first and second portions are complementary but may not be completely symmetrical, as the hairpin structure may contain 3′ or 5′ overhang nucleotides (e.g., a 1, 2, 3, 4, or 5 nucleotide overhang). The RNA molecules of the invention can be expressed from a vector or produced chemically or synthetically.

### 5.2.3 Inhibitor-Cell Penetrating Peptide Conjugates

In certain embodiments of the instant invention, the caspase-9 signaling pathway inhibitor is conjugated to a cell penetrating peptide to form an inhibitor-cell penetrating peptide conjugate. The conjugate can facilitate delivery of the inhibitor to into a cell in which it is desirable to prevent apoptosis.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. In certain embodiments, the cell-penetrating peptide used in the membrane-permeable complex of the present invention preferably comprises at least one non-functional cysteine residue, which is either free or derivatized to form a disulfide link with the caspase-9 signaling pathway inhibitor, which has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, Penetratin1, transportan, pIsl, TAT(48-60), pVEC, MTS, and MAP.

The cell-penetrating peptides of the present invention include those sequences that retain certain structural and functional features of the identified cell-penetrating peptides, yet differ from the identified peptides' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions, as described above in connection with polypeptide caspase-9 signaling pathway inhibitors. In certain embodiments, a cell-penetrating peptide of the present invention is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the identified peptide and is capable of mediating cell penetration. The effect of the amino acid substitutions on the ability of the synthesized peptide to mediate cell penetration can be tested using the methods disclosed in Examples section, below.

In certain embodiments of the present invention, the cell-penetrating peptide of the membrane-permeable complex is Penetratin1, comprising the peptide sequence RQIKIWFQNRRMKWKK (SEQ ID NO: 2), or a conservative variant thereof. As used herein, a “conservative variant” is a peptide having one or more amino acid substitutions, wherein the substitutions do not adversely affect the shape—or, therefore, the biological activity (i.e., transport activity) or membrane toxicity—of the cell-penetrating peptide.

Penetratin1 is a 16-amino-acid polypeptide derived from the third alpha-helix of the homeodomain of Drosophila antennapedia. Its structure and function have been well studied and characterized: Derossi et al., Trends Cell Biol., 8(2):84-87, 1998; Dunican et al., Biopolymers, 60(1):45-60, 2001; Hallbrink et al., Biochim. Biophys. Acta, 1515(2):101-09, 2001; Bolton et al., Eur. J. Neurosci., 12(8):2847-55, 2000; Kilk et al., Bioconjug. Chem., 12(6):911-16, 2001; Bellet-Amalric et al., Biochim. Biophys. Acta, 1467(1):131-43, 2000; Fischer et al., J. Pept. Res., 55(2): 163-72, 2000; Thoren et al., FEBS Lett., 482(3):265-68, 2000.

It has been shown that Penetratin1 efficiently carries avidin, a 63-kDa protein, into human Bowes melanoma cells (Kilk et al., Bioconjug. Chem., 12(6):911-16, 2001). Additionally, it has been shown that the transportation of penetratin and its cargo is non-endocytotic and energy-independent, and does not depend upon receptor molecules or transporter molecules. Furthermore, it is known that penetratin is able to cross a pure lipid bilayer (Thoren et al., FEBS Lett., 482(3):265-68, 2000). This feature enables Penetratin1 to transport its cargo, free from the limitation of cell-surface-receptor/-transporter availability. The delivery vector previously has been shown to enter all cell types (Derossi et al., Trends Cell Biol., 8(2):84-87, 1998), and effectively to deliver peptides (Troy et al., Proc. Natl. Acad. Sci. USA, 93:5635-40, 1996) or antisense oligonucleotides (Troy et al., J. Neurosci., 16:253-61, 1996; Troy et al., J. Neurosci., 17:1911-18, 1997).

Other non-limiting embodiments of the present invention involve the use of the following exemplary cell permeant molecules: RL16 (H-RRLRRLLRRLLRRLRR-OH) (SEQ ID NO: 3), a sequence derived from Penetratin1 with slightly different physical properties (Biochim Biophys Acta. 2008 July-August; 1780(7-8):948-59); and RVG-RRRRRRRRR (SEQ ID NO: 4), a rabies virus sequence which targets neurons see P. Kumar, H. Wu, J. L. McBride, K. E. Jung, M. H. Kim, B. L. Davidson, S. K. Lee, P. Shankar and N. Manjunath, Transvascular delivery of small interfering RNA to the central nervous system, Nature 448 (2007), pp. 39-43.

In certain alternative non-limiting embodiments of the present invention, the cell-penetrating peptide of the membrane-permeable complex is a cell-penetrating peptides selected from the group consisting of: transportan, pIS1, Tat(48-60), pVEC, MAP, and MTS. Transportan is a 27-amino-acid long peptide containing 12 functional amino acids from the amino terminus of the neuropeptide galanin, and the 14-residue sequence of mastoparan in the carboxyl terminus, connected by a lysine (Pooga et al., FASEB J., 12(1):67-77, 1998). It comprises the amino acid sequence GWTLNSAGYLLGKINLKALAALAKKIL (SEQ ID NO: 5), or a conservative variant thereof.

pIsl is derived from the third helix of the homeodomain of the rat insulin 1 gene enhancer protein (Magzoub et al., Biochim. Biophys. Acta, 1512(1):77-89, 2001; Kilk et al., Bioconjug. Chem., 12(6):911-16, 2001). pIsl comprises the amino acid sequence PVIRVW FQNKRCKDKK (SEQ ID NO: 6), or a conservative variant thereof.

Tat is a transcription activating factor, of 86-102 amino acids, that allows translocation across the plasma membrane of an HIV-infected cell, to transactivate the viral genome (Hallbrink et al., Biochem. Biophys. Acta., 1515(2):101-09, 2001; Suzuki et al., J. Biol. Chem., 277(4):2437-43, 2002; Futaki et al., J. Biol. Chem., 276(8):5836-40, 2001). A small Tat fragment, extending from residues 48-60, has been determined to be responsible for nuclear import (Vives et al., J. Biol. Chem., 272(25):16010-017, 1997); it comprises the amino acid sequence GRKKRRQRRRPPQ (SEQ ID NO: 7), or a conservative variant thereof.

pVEC is an 18-amino-acid-long peptide derived from the murine sequence of the cell-adhesion molecule, vascular endothelial cadherin, extending from amino acid 615-632 (Elmquist et al., Exp. Cell Res., 269(2):237-44, 2001). pVEC comprises the amino acid sequence LLIILRRRIRKQAHAH (SEQ ID NO: 8), or a conservative variant thereof.

MTSs, or membrane translocating sequences, are those portions of certain peptides which are recognized by the acceptor proteins that are responsible for directing nascent translation products into the appropriate cellular organelles for further processing (Lindgren et al., Trends in Pharmacological Sciences, 21(3):99-103, 2000; Brodsky, J. L., Int. Rev. Cyt., 178:277-328, 1998; Zhao et al., J. Immunol. Methods, 254(1-2):137-45, 2001). An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides (Lindgren et al., Trends in Pharmacological Sciences, 21(3):99-103, 2000; Morris et al., Nucleic Acids Res., 25:2730-36, 1997). MPS comprises the amino acid sequence GALFLGWLGAAGSTMGAWSQPKKKRKV (SEQ ID NO: 9), or a conservative variant thereof.

Model amphipathic peptides, or MAPs, form a group of peptides that have, as their essential features, helical amphipathicity and a length of at least four complete helical turns (Scheller et al., J. Peptide Science, 5(4):185-94, 1999; Hallbrink et al., Biochim. Biophys. Acta., 1515(2):101-09, 2001). An exemplary MAP comprises the amino acid sequence KLALKLALKALKAALKLA-amide, or a conservative variant thereof.

In certain embodiments, the cell-penetrating peptides and the caspase-9 signaling pathway inhibitors described above are covalently bound to form conjugates. In certain embodiments the cell-penetrating peptide is operably linked to a peptide caspase-9 signaling pathway inhibitor via recombinant DNA technology. For example, in embodiments where the caspase-9 signaling pathway inhibitor is a peptide or polypeptide sequence, a nucleic acid sequence encoding that caspase-9 signaling pathway inhibitor can be introduced either upstream (for linkage to the amino terminus of the cell-penetrating peptide) or downstream (for linkage to the carboxy terminus of the cell-penetrating peptide), or both, of a nucleic acid sequence encoding the caspase-9 signaling pathway inhibitor of interest. Such fusion sequences comprising both the caspase-9 signaling pathway inhibitor encoding nucleic acid sequence and the cell-penetrating peptide encoding nucleic acid sequence can be expressed using techniques well known in the art.

In certain embodiments the caspase-9 signaling pathway inhibitor can be operably linked to the cell-penetrating peptide via a non-covalent linkage. In certain embodiments such non-covalent linkage is mediated by ionic interactions, hydrophobic interactions, hydrogen bonds, or van der Waals forces.

In certain embodiments the caspase-9 signaling pathway inhibitor is operably linked to the cell penetrating peptide via a chemical linker. Examples of such linkages typically incorporate 1-30 nonhydrogen atoms selected from the group consisting of C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In certain embodiments, the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. In certain embodiments, the linker is a combination of single carbon-carbon bonds and carboxamide, sulfonamide or thioether bonds.

A general strategy for conjugation involves preparing the cell-penetrating peptide and the caspase-9 signaling pathway inhibitor components separately, wherein each is modified or derivatized with appropriate reactive groups to allow for linkage between the two. The modified the caspase-9 signaling pathway inhibitor is then incubated together with a cell-penetrating peptide that is prepared for linkage, for a sufficient time (and under such appropriate conditions of temperature, pH, molar ratio, etc.) as to generate a covalent bond between the cell-penetrating peptide and the caspase-9 signaling pathway inhibitor molecule.

Numerous methods and strategies of conjugation will be readily apparent to one of ordinary skill in the art, as will the conditions required for efficient conjugation. By way of example only, one such strategy for conjugation is described below, although other techniques, such as the production of fusion proteins or the use of chemical linkers is within the scope of the instant invention.

In certain embodiments, when generating a disulfide bond between the caspase-9 signaling pathway inhibitor molecule and the cell-penetrating peptide of the present invention, the caspase-9 signaling pathway inhibitor molecule can be modified to contain a thiol group, and a nitropyridyl leaving group can be manufactured on a cysteine residue of the cell-penetrating peptide. Any suitable bond (e.g., thioester bonds, thioether bonds, carbamate bonds, etc.) can be created according to methods generally and well known in the art. Both the derivatized or modified cell-penetrating peptide, and the modified the caspase-9 signaling pathway inhibitor are reconstituted in RNase/DNase sterile water, and then added to each other in amounts appropriate for conjugation (e.g., equimolar amounts). The conjugation mixture is then incubated for 60 min at 37° C., and then stored at 4° C. Linkage can be checked by running the vector-linked caspase-9 signaling pathway inhibitor molecule, and an aliquot that has been reduced with DTT, on a 15% non-denaturing PAGE. Caspase-9 signaling pathway inhibitor molecules can then be visualized with the appropriate stain.

In certain embodiments, the conjugates of the present invention will comprise a double stranded nucleic acid conjugated to a cell-penetrating peptide. In the practice of certain of such embodiments, at least one strand of the double-stranded ribonucleic acid molecule (either the sense or the antisense strand) may be modified for linkage with a cell-penetrating peptide (e.g., with a thiol group), so that the covalent bond links the modified strand to the cell-penetrating peptide. Where the strand is modified with a thiol group, the covalent bond linking the cell-penetrating peptide and the modified strand of the ribonucleic acid molecule can be a disulfide bond, as is the case where the cell-penetrating peptide has a free thiol function (i.e., pyridyl disulfide or a free cysteine residue) for coupling. However, it will be apparent to those skilled in the art that a wide variety of functional groups may be used in the modification of the ribonucleic acid, so that a wide variety of covalent bonds (e.g., ester bonds, carbamate bonds, sulfonate bonds, etc.) may be applicable. Additionally, the membrane-permeable complex of the present invention may further comprise a moiety conferring target-cell specificity to the complex.

In certain embodiments, the present invention is directed to a Penetratin1 1-XBIR3 conjugate. In certain of such embodiments, the sequence of the Penetratin1 1-XBIR3 sequence is PENT-XBIR3: RQIKIWFQNRRMKWKK-s-s-NTLPRNPSMADYEARIFTFGTWIYSVNKEQLARAGFYALGEGDKVKCFHCGGGL TDWRPSEDPWEQHARWYPGCRYLLEQRGQEYINNIHLTHS (SEQ ID NOS 2 and 11, respectively).

### 5.2.3 Pharmaceutical Compositions

In certain embodiments, the caspase-9 signaling pathway inhibitors or membrane-permeable complexes of the instant invention are formulated for nasal administration. For nasal administration, solutions or suspensions comprising the caspase-9 signaling pathway inhibitors or membrane-permeable complexes of the instant invention can be formulated for direct application to the nasal cavity by conventional means, for example with a dropper, pipette or spray. Other means for delivering the nasal spray composition, such as inhalation via a metered dose inhaler (MDI), may also be used according to the present invention. Several types of MDIs are regularly used for administration by inhalation. These types of devices can include breath-actuated MDI, dry powder inhaler (DPI), spacer/holding chambers in combination with MDI, and nebulizers. The term “MDI” as used herein refers to an inhalation delivery system comprising, for example, a canister containing an active agent dissolved or suspended in a propellant optionally with one or more excipients, a metered dose valve, an actuator, and a mouthpiece. The canister is usually filled with a solution or suspension of an active agent, such as the nasal spray composition, and a propellant, such as one or more hydrofluoroalkanes. When the actuator is depressed a metered dose of the solution is aerosolized for inhalation. Particles comprising the active agent are propelled toward the mouthpiece where they may then be inhaled by a subject. The formulations may be provided in single or multidose form. For example, in the case of a dropper or pipette, this may be achieved by the patient administering an appropriate, predetermined volume of the solution or suspension. In the case of a spray, this may be achieved for example by means of a metering atomising spray pump. To improve nasal delivery and retention, the compositions according to the invention may be encapsulated with cyclodextrins, or formulated with agents expected to enhance delivery and retention in the nasal mucosa.

Commercially available administration devices that are used or can be adapted for nasal administration of a composition of the invention include the AERONEB™ (Aerogen, San Francisco, Calif.), AERONEB GO™ (Aerogen); PARI LC PLUS™, PARI BOY™ N, PARI™ eflow (a nebulizer disclosed in U.S. Pat. No. 6,962,151), PARI LC SINUS™, PARI SINUSTAR™., PARI SINUNEB™, VibrENT™ and PARI DURANEB™ (PARI Respiratory Equipment, Inc., Monterey, Calif. or Munich, Germany); MICROAIR™ (Omron Healthcare, Inc, Vernon Hills, Ill.), HALOLITE™ (Profile Therapeutics Inc, Boston, Mass.), RESPIMAT™ (Boehringer Ingelheim, Germany), AERODOSE™ (Aerogen, Inc, Mountain View, Calif.), OMRON ELITE™ (Omron Healthcare, Inc, Vernon Hills, Ill.), OMRON MICROAIR™ (Omron Healthcare, Inc, Vernon Hills, Ill.), MABISMIST™ II (Mabis Healthcare, Inc, Lake Forest, Ill.), LUMISCOPE™ 6610, (The Lumiscope Company, Inc, East Brunswick, N.J.), AIRSEP MYSTIQUE™, (AirSep Corporation, Buffalo, N.Y.), ACORN-1™ and ACORN-II™ (Vital Signs, Inc, Totowa, N.J.), AQUATOWER™ (Medical Industries America, Adel, Iowa), AVA-NEB™ (Hudson Respiratory Care Incorporated, Temecula, Calif.), AEROCURRENT™ utilizing the AEROCELL™ disposable cartridge (AerovectRx Corporation, Atlanta, Ga.), CIRRUS™ (Intersurgical Incorporated, Liverpool, N.Y.), DART™ (Professional Medical Products, Greenwood, S.C.), DEVILBISS™ PULMO AIDE (DeVilbiss Corp; Somerset, Pa.), DOWNDRAFT™ (Marquest, Englewood, Colo.), FAN JET™ (Marquest, Englewood, Colo.), MB-5™ (Mefar, Bovezzo, Italy), MISTY NEB™ (Baxter, Valencia, Calif.), SALTER 8900™ (Salter Labs, Arvin, Calif.), SIDESTREAM™ (Medic-Aid, Sussex, UK), UPDRAFT-II™ (Hudson Respiratory Care; Temecula, Calif.), WHISPER JET™ (Marquest Medical Products, Englewood, Colo.), AIOLOS™ (Aiolos Medicnnsk Teknik, Karlstad, Sweden), INSPIRON™ (Intertech Resources, Inc., Bannockburn, Ill.), OPTIMIST™ (Unomedical Inc., McAllen, Tex.), PRODOMO™, SPIRA™ (Respiratory Care Center, Hameenlinna, Finland), AERx™ Essence™ and Ultra™, (Aradigm Corporation, Hayward, Calif.), SONIK™ LDI Nebulizer (Evit Labs, Sacramento, Calif.), ACCUSPRAY™ (BD Medical, Franklin Lake, N.J.), ViaNase ID™ (electronic atomizer; Kurve, Bothell, Wash.), OptiMist™ device or OPTINOSE™ (Oslo, Norway), MAD Nasal™ (Wolfe Tory Medical, Inc., Salt Lake City, Utah), Freepod™ (Valois, Marly be Roi, France), Dolphin™ (Valois), Monopowder™ (Valois), Equadel™ (Valois), VP3™ and VP7™ (Valois), VP6 Pump™ (Valois), Standard Systems Pumps™ (Ing. Erich Pfeiffer, Radolfzell, Germany), AmPump™ (Ing. Erich Pfeiffer), Counting Pump™ (Ing. Erich Pfeiffer), Advanced Preservative Free System™ (Ing. Erich Pfeiffer), Unit Dose System™ (Ing. Erich Pfeiffer), Bidose System™ (Ing. Erich Pfeiffer), Bidose Powder System™ (Ing. Erich Pfeiffer), Sinus Science™ (Aerosol Science Laboratories, Inc., Camarillo, Calif.), ChiSys™ (Archimedes, Reading, UK), Fit-Lizer™ (Bioactis, Ltd, a SNBL subsidiary (Tokyo, J P), Swordfish V™ (Mystic Pharmaceuticals, Austin, Tex.), DirectHaler™ Nasal (DirectHaler, Copenhagen, Denmark) and SWIRLER™ Radioaerosol System (AMICI, Inc., Spring City, Pa.).

To facilitate delivery to a cell, tissue, or subject, the caspase-9 signaling pathway inhibitor, or membrane-permeable conjugates thereof, of the present invention may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the caspase-9 signaling pathway inhibitor or membrane-permeable conjugates or the biological activity of the recipient of the composition. Suitable pharmaceutical carriers, excipients, and/or diluents for use in the present invention include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water. Specific formulations of compounds for therapeutic treatment are discussed in Hoover, J. E., Remington's Pharmaceutical Sciences (Easton, Pa.: Mack Publishing Co., 1975) and Liberman and Lachman, eds., Pharmaceutical Dosage Forms (New York, N.Y.: Marcel Decker Publishers, 1980).

In accordance with the methods of the present invention, the quantity of the caspase-9 signaling pathway inhibitor or membrane-permeable conjugates thereof that is administered to a cell, tissue, or subject should be an amount that is effective to inhibit the caspase-9 signaling pathway member within the tissue or subject. This amount is readily determined by the practitioner skilled in the art. The specific dosage employed in connection with any particular embodiment of the present invention will depend upon a number of factors, including the type inhibitor used, the caspase-9 signaling pathway member to be inhibited, and the cell type expressing the target. Quantities will be adjusted for the body weight of the subject, and the particular disease or condition being targeted.

### 6. Examples

### 6.1 Caspases, Cerebral Ischemia and Edema

For more than a decade, members of the caspase family of death proteases have been implicated in cerebral ischemia and neurodegeneration. Troy, et al., Prog Mol Biol Transl Sci 99, 265-305 (2011). Yet, identifying specific caspase pathways has proven arduous. Caspases are a multi-membered family of cell death proteases. The study of effects of specific caspases has been hampered by the use of reagents (tetra and pentapeptide inhibitors/substrates) which were designed as tools to target specific individual caspases but have been shown to actually target multiple caspases. McStay et al., Cell Death Differ (2007).

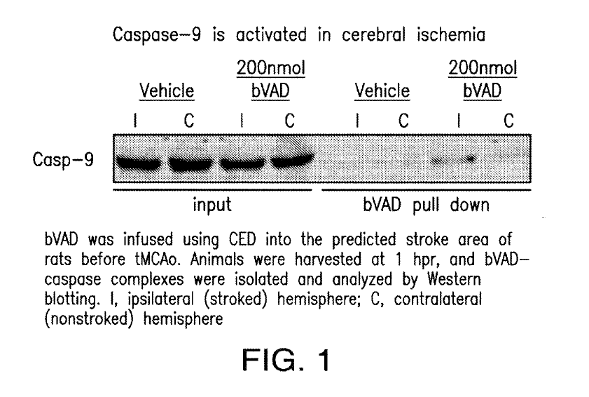

In order to determine which caspases were actually responsible for neuronal degeneration in stroke, a caspase affinity ligand was employed (Tu et al., Nat Cell Biol 8 (1), 72-77 (2006)) and developed an unbiased in vivo approach to detect the proximal caspases activated in stroke (Akpan et al., The Journal of Neuroscience: the official journal of the Society for Neuroscience 31 (24), 8894-8904 (2011)). Rats were treated with the caspase affinity ligand, bVAD, delivered to the brain parenchmya by convection enhanced delivery (CED) prior to onset of occlusion. In this way the caspase affinity ligand will bind to the first caspase activated by the ischemic insult. After 2 hrs of occlusion and 1 hr reperfusion animals were harvested and bVAD-caspase complexes isolated with streptavidin, and active caspase-9 identified by western blot (FIG. 1).

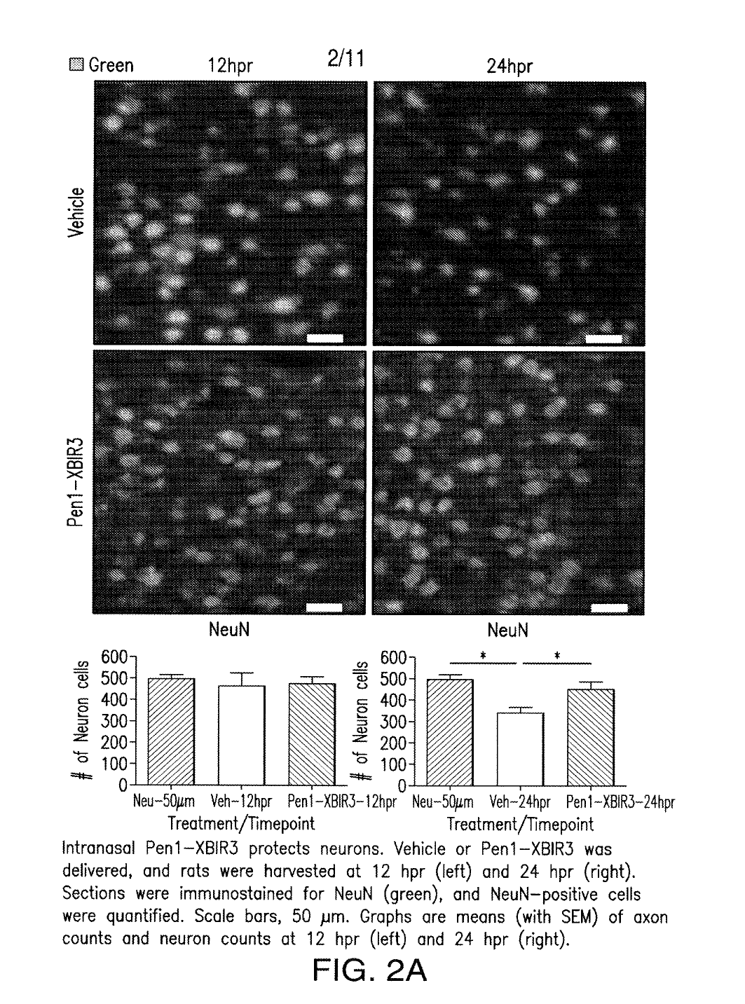

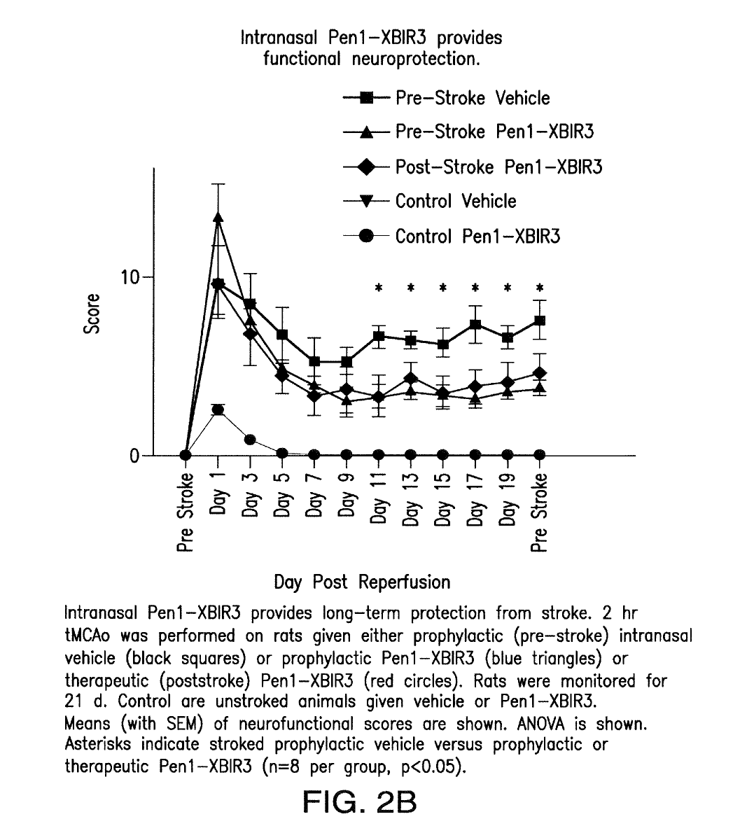

This novel approach showed that caspase-9 was the key initiator caspase regulating neurodegeneration induced by cerebral ischemia. Akpan et al., The Journal of Neuroscience: the official journal of the Society for Neuroscience 31 (24), 8894-8904 (2011). Caspase-9 activates caspase-6 in neuronal soma and processes and this cascade is critical in the progression of stroke. Further a cell permeant caspase-9 specific inhibitor (Pen1-XBIR3) has been devised. This inhibitor is derived from the BIR3 domain of the endogenous caspase inhibitor XIAP; BIR3 is a specific inhibitor of caspase-9 activity. Linking the XBIR3 domain to the cell penetrating protein, Penetratin1 (Pen1) provided intracellular delivery. Intranasal delivery of this inhibitor prophylactically or therapeutically provided substantial cellular and functional neuroprotection (FIG. 2).

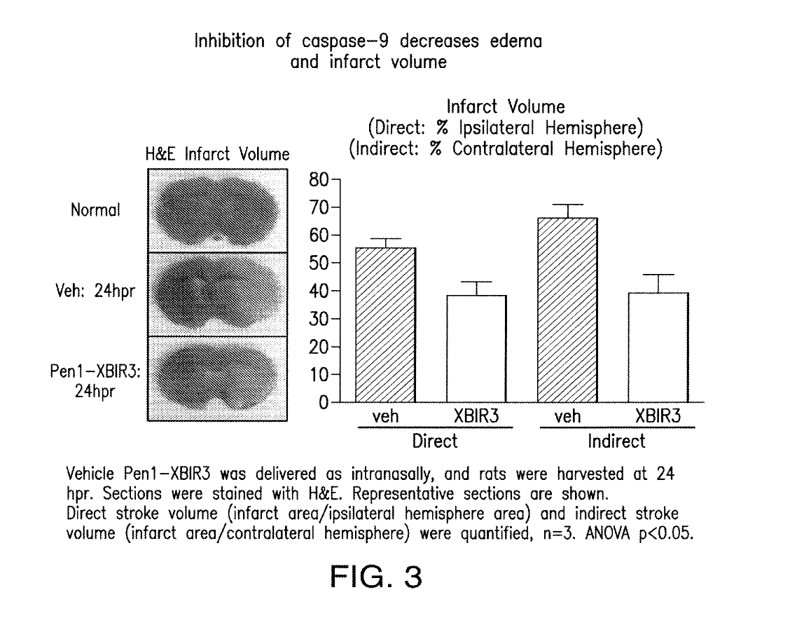

Surprisingly, the instant data also showed that the caspase-9 specific inhibitor inhibited the development of edema. FIG. 3 shows representative brain sections stained with H&E. Quantification of the staining was performed for direct and indirect stroke volumes. Direct stroke volume, measured as infarct area relative to the ipsilateral hemisphere area, automatically corrects the size of the infarct for its level of edema. Indirect stroke volume, on the other hand, is measured as infarct area relative to contralateral hemisphere and does not account for the contribution of edema to infarct size. The graph in FIG. 3 shows that for vehicle-treated animals, indirect stroke volume showed approximately an 11% expansion in the size of the infarct, indicative of edema in this cohort. For the Pen1-XBIR3 cohort, stroke volume was essentially identical between direct and indirect measurements. This finding indicates that Pen1-XBIR3 inhibition of caspase-9 activity reduces edema during tMCAo, in addition to decreasing stroke volume.

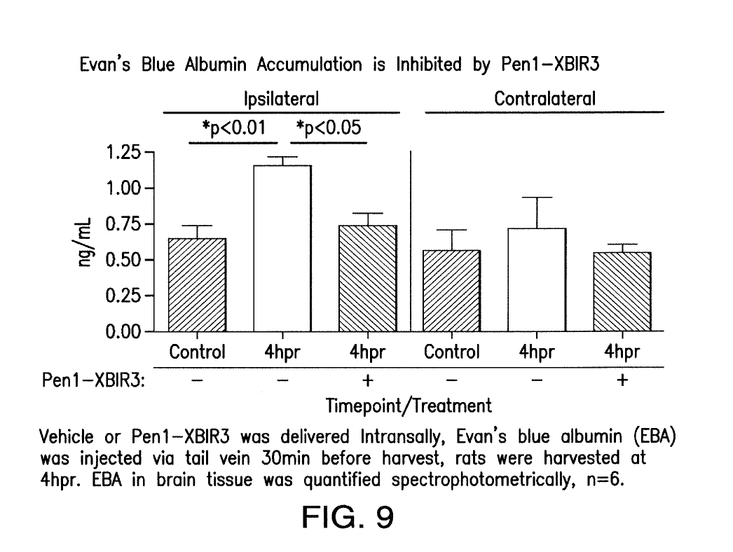

The studies of edema have been extended utilizing Evan's Blue albumin (EBA) injection to measure vasogenic edema. EBA only crosses into the brain when the BBB is breached. As shown in FIG. 9, tMCAo induces EBA accumulation by 4 hpr (6 hrs total) showing that there is extravasation of the dye into the brain; this is blocked by intranasal administration of Pen1-XBIR3, providing additional support for a role for caspase-9 in vasogenic edema.

Caspases have been linked with edema in studies using endothelial cell lines. It has been proposed that the activation of MMPs during ischemia leads to the activation of caspases and that both can participate in edema. Lee et al., Journal of cerebral blood flow and metabolism: official journal of the International Society of Cerebral Blood Flow and Metabolism 24 (7), 720-727 (2004). Work using a cell culture model for the neurovascular unit shows that DEVD (SEQ ID NO: 12) can partially inhibit cleavage of ZO-1 and claudin-5, suggesting a function for caspases in edema (Zehendner et al., PLoS One 6 (2), e16760 (2011)); DEVD (SEQ ID NO: 12) can inhibit multiple caspases (McStay et al., Cell Death Differ (2007)). Specific inhibition of caspase-9 activity 6 hours after occlusion provides functional neuroprotection out to 3 weeks (FIG. 2). A key event that is proposed to have been blocked when Pen1-XBIR3 was delivered at 6 hours is caspase-9 dependent formation of edema and that inhibition of this activity reduces many of the adverse sequelae of stroke. The data using EBA (FIG. 9) supports this, as there is EBA extravasation at this time point and it is blocked by Pen1-XBIR3. As a first step in determining how caspase-9 activity regulates edema, whether caspase-9 is found in the small BVs which are the source of the edema was examined.

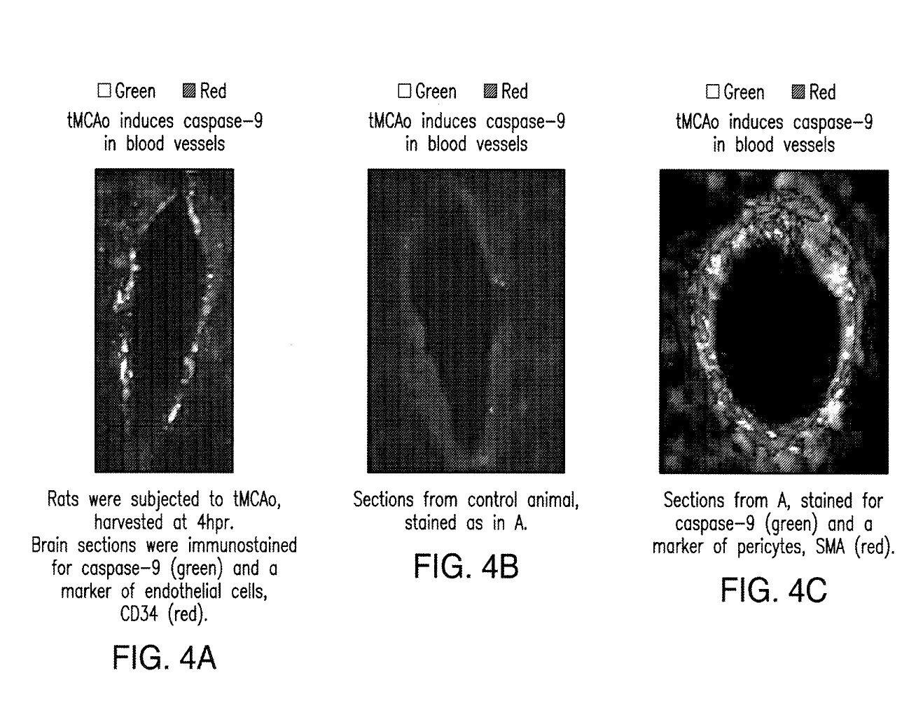

Whether caspase-9 is found in the small blood vessels which are the source of the edema was further examined. Using IHC, caspase-9 was stained for and markers of pericytes (SMA) or endothelial cells (CD34). These data (FIG. 4) show that there is an increase in caspase-9 in small blood vessels after ischemia (compare FIGS. 4A and B), with expression of caspase-9 mainly in the endothelial cells (panel A), with a subset of pericytes showing some caspase-9 expression (FIG. 4C).

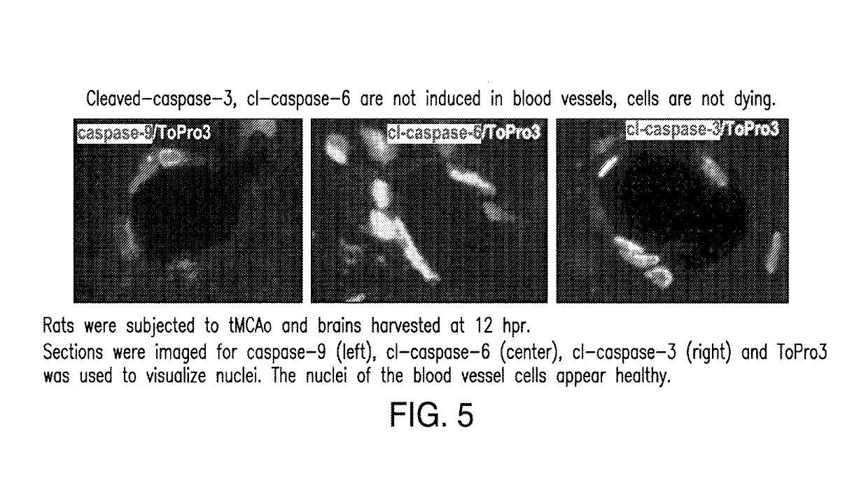

To assess whether the blood vessel cells were dying, ToPro3 staining was used to evaluate nuclear morphology and nuclei appear healthy, shown in FIG. 5. These sections were also stained for cl-casp3 and cl-casp6, downstream effectors of caspase-9 (Troy et al., J Biol Chem 277, 34295-34302 (2002)), to see if these caspases were activated in the blood vessels and found no increase in either, while there is an increase in caspase-9 in the same brain (FIG. 5). These data support the position that caspase-9 is acting in a non-death pathway in the blood vessels to promote edema.

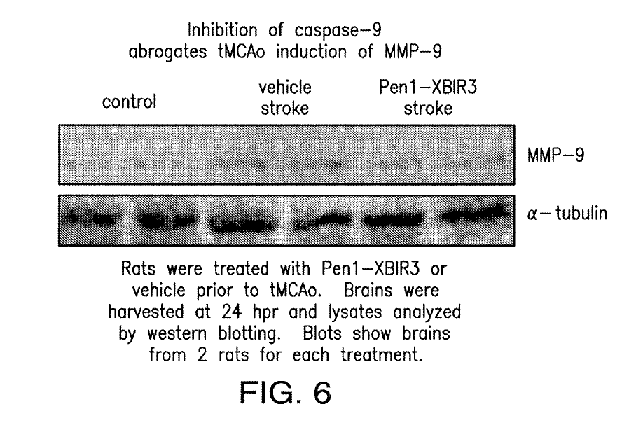

As noted above, MMP-9 is increased in cerebral edema and knockout of MMP-9 provides protection from edema. To determine if caspase-9 and MMP-9 are acting in the same pathway levels of MMP-9 protein from animals subjected to tMCAo with and without inhibition of caspase-9 were examined and it was found that the caspase-9 inhibitor Pen1-XBIR3 abrogated the 3-fold increase of MMP-9 induced by tMCAo (FIG. 6), suggesting that caspase-9 acts upstream of MMP-9. MMP-9 is regulated by its endogenous inhibitor, TIMP-1, which has a potential caspase-9 cleavage site (Table 1). Levels of TIMP-1 decrease during stroke. Wu et al., J Mol Neurosci (2011). Caspase-9 activity is therefore proposed to directly lead to the development of edema through cleavage of selective substrates in the blood vessels, including TIMP-1. Table 1 shows that several of the proteins that mediate blood vessel integrity are potential caspase-9 substrates. Cleavage of TIMP-1 would lead to a release of MMP-9 inhibition and an increase in MMP-9, which would then proceed to cleave vascular substrates too.

| | Caspase-9 amino | |
|---|---|---|
| Protein | acid cleavage site(s) | Fragment size(s) |
| Aquaporin-4 | 370 | 40/1 |
| ZO-1 | 1382 | 152/46 |
| | 577 | 63/135 |
| Claudin 3 | none | |
| Claudin 5 | none | |
| Claudin 12 | none | |
| Occludin | 471 | 51/11 |
| 7H6 (barmotin) | 457 | 50/85 |
| cingulin | 875 | 96/58 |
| | 713 | 78/76 |
| AF-6 (afadin) | 184 | 20/189 |
| | 1637 | 180/29 |
| PECAM-1 | 500 | 55/30 |
| JAM-1 | 239 | 26/15 |
| TIMP-1 | 248 | 27/5 |

### 6.2 p75NTR Activation of Caspase-9

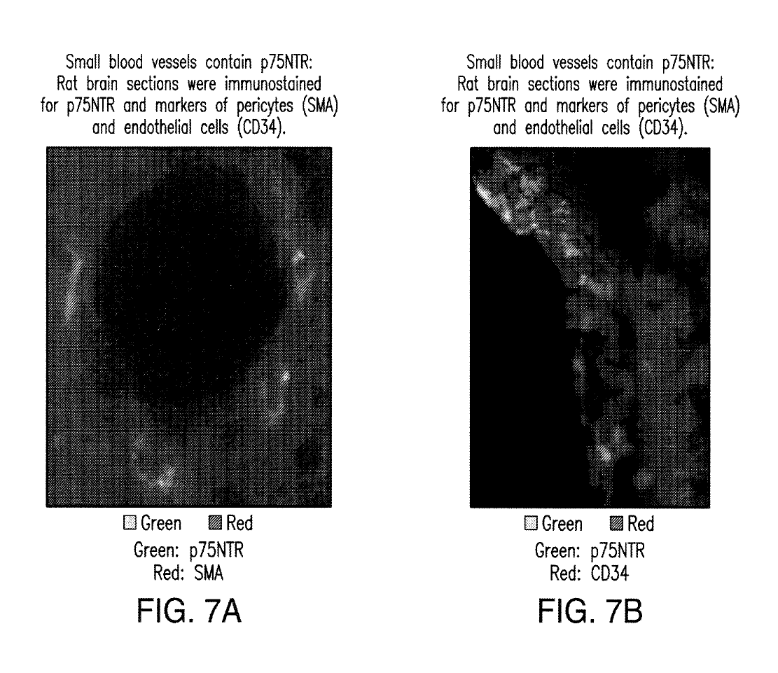

Further support for the therapeutic strategies outlined herein is provided by the presence of the machinery to activate caspase-9 in the blood vessels. It has been shown that caspase-9 can be activated by neurotrophin signaling though the p75 neurotrophin receptor, p75NTR. Troy et al., J Biol Chem 277, 34295-34302 (2002). Prior studies show that p75NTR is present on pericytes. Diaz-Flores et al., Histology and histopathology 24 (7), 909-969 (2009) and Meinecke et al., Neuroscience 54 (1), 105-116 (1993). Data presented herein confirm this finding (FIG. 7A) and now show evidence of p75NTR on endothelial cells in small blood vessels (FIG. 7B).

Since the initial identification of p75NTR as a neurotrophin receptor and the subsequent identification of the Trk family of receptors as the high affinity neurotrophin receptors, it has been found that the high affinity ligands for p75NTR are the proneurotrophins Teng et al., Dev Neurobiol 70 (5), 350-359 (2010). As with many peptides, neurotrophins are synthesized as prepropeptides, which are processed to proneurotrophins (proNTs) and finally to the mature NTs. Under normal conditions, processing occurs intracellularly, and is mediated by furins or proconvertases. The mature neurotrophins are secreted, bind to, and signal via their cognate Trk receptors. Reichardt, Philos Trans R Soc Lond B Biol Sci 361 (1473), 1545-1564 (2006). Under stress conditions, such as seizures, proNTs are secreted and bind to p75NTR to signal death via activation of caspases-9, -6 and -3. Troy et al., J Biol Chem 277, 34295-34302 (2002). ProNT can be cleaved in the extracellular milieu by matrix metalloproteases (MMPs). W. Friedman has shown that secreted proNGF mediates neuronal degeneration via p75NTR signaling in an in vivo seizure model, and that the increase in proNGF correlates with a decrease in MMP-7 activity. In addition, this study showed that administration of exogenous MMP-7 decreased proNGF levels and neuronal degeneration. Le et al., The Journal of Neuroscience: the official journal of the Society for Neuroscience 32 (2), 703-712 (2012).

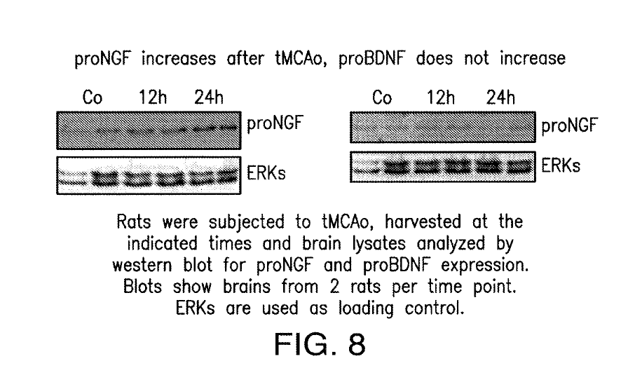

Evidence is herein provided that proNGF levels increase in stroke, but proBDNF levels do not change (FIG. 8). Densitometry shows that proNGF levels increase by 50% at 12 hours and 80% at 24 hours; proBDNF levels did not change over this time course. This supports the position that the proNGF-p75NTR-caspase-9 signaling pathway is critical for edema. Similarly, changes in p75NTR expression have been reported in ischemia induced by cortical devascularization (Angelo et al., Journal of Neuroscience Research 87 (8), 1892-1903 (2009)) and in a model of hypo-osmolar stress (Ramos et al., The Journal of Neuroscience: the official journal of the Society for Neuroscience 27 (6), 1498-1506 (2007)).

### 6.3 Induction of proNGF Triggers Caspase-9 Activation

The studies described herein show that a specific caspase-9 inhibitor, Pen1-XBIR3, abrogates edema induced by cerebral ischemia in a rodent model of stroke (tMCAo). Caspase-9 activation can be induced by proneurotrophin binding to p75NTR. In addition, proNGF, but not proBDNF, levels are increased after onset of stroke, which supports the position that caspase-9 activation is mediated by proNGF acting through p75NTR on the small blood vessels. Thus, manipulating proNGF levels should effect caspase-9 activation and edema. proNGF is also of interest, as the data show no changes in proBDNF, and proNGF has been closely linked to neurodegeneration.

Data presented herein indicates that proNGF is increased in stroke penumbra lysates at 12 and 24 hours post-reperfusion (hpr) (FIG. 8). Edema can be morphologically detected by 12 hpr and continues over 2-3 days. To ascertain that proNGF induction triggers caspase-9 activation, proNGF protein levels and NGF RNA levels in tissue lysates (core and penumbra) will be determined from mice at 0 h, 1 h, 4 h, 8 h, 12 h, and 24 h post-reperfusion. 8 animals will be used per time point, 4 for protein, 4 for mRNA. The study will also include 8 sham (animals subjected to everything except occlusion) animals and 8 non-stroked controls. The distribution of pro-NGF will be also be examined immunohistochemically using an antibody that detects proNGF but not mature NGF (Le et al., The Journal of neuroscience: the official journal of the Society for Neuroscience 32 (2), 703-712 (2012)) and also markers for pericytes, endothelial cells, neurons, astrocytes, and microglia will be stained for in order to determine the cellular location of proNGF. These studies will also use 4 animals per time point, shams and non-stroked controls. To assay edema, MRI and H & E staining will be employed and cerebral edema will be assayed in terms of the accumulation of Evans Blue-albumin (EBA). This approach is well developed for studies of pulmonary edema. Briefly, EBA will be injected intravenously. After 2 hours, EBA concentration will be assayed by spectrophotometry in samples of brain tissue obtained from different sites. Increase in EBA concentration will denote increased protein extravasation, providing regional quantification of edema formation. The levels of edema markers (MMP-9, Aquaporin-4), and of the BBB marker SMI-71 will also be determined biochemically and by IHC.

### 6.4 Activation of Caspase-9 by Exogenous Cleavage-Resistance proNGF

If proNGF is critical in the development of edema, treating cultured blood vessel cells with cleavage-resistant proNGF should be sufficient to activate caspase-9 in blood vessels. Pericytes and endothelial cells will be isolated from the brain as outlined in Quadri et al., Am J Physiol Lung Cell Mol Physiol 292 (1), L334-342 (2007) and Katyshev et al., Methods in molecular biology 814, 467-481 (2012). The primary cultures will be treated with cleavage-resistant proNGF (provided by B. Hempstead) to determine if caspase-9 is activated. For these studies, isolated blood vessels cells will be pretreated with bVAD for 2 hrs, then treated with proNGF, and harvested for isolation of active caspases at 30 min, 1 h, 2 h and 4 h, as previously described (Tizon et al., J Alzheimers Dis 19 (3), 885-894 (2010)), see FIG. 1. In parallel, a line of rat brain endothelial cells, RBE cells, which have been shown to contain tight junction proteins and form endothelial barriers, will be used (He et al., Neuroscience 188, 35-47 (2011) and Brown et al., Brain research 1130 (1), 17-30 (2007)) to express p75NTR32. These cells will be cultured for 10 days prior to experiments to ensure that tight junctions have formed in culture. As a read-out for edema-like changes in culture, transmembrane electrical resistance (TER) will be measured. TER will be measured prior to treatment of cells and after treatment with cleavage-resistant proNGF. Integrity of tight junctions in the cultured cells will also be determined by immunocytochemistry for ZO-1, a component of the tight junction, and measuring the border staining of cells with ZO-1 as described in Simon et al., Ann Biomed Eng 39 (1), 394-401 (2011). Activation of caspase-9 will be determined as described for the primary cultures.

### 6.5 MMP-7 Level & Activity During Cerebral Ischemia

MMP-7 can cleave proNGF24. In a seizure model MMP-7 levels decrease over time as proNGF levels increase, which supports a function for MMP-7 in processing proNGF in the brain. Le et al., The Journal of neuroscience: the official journal of the Society for Neuroscience 32 (2), 703-712 (2012). MMP-7 levels will be measured with western blotting using the lysates from the proNGF time course. The distribution of MMP-7 activity will be assayed in the brain sections from the tMCAO time course experiment described herein using fluorescent zymography for MMP-7 activity.

### 6.6 Exogenous MMP-7 Converts proNGF to NGF to Decrease Caspase-9 Activation, and Reduce Edema

Because MMP-7 can cleave proNGF, administering exogenous MMP-7 should decrease the levels of proNGF, decrease caspase-9 in blood vessels, and reduce edema. MMP-7 (1 μg) will be administered by CED into the striatum prior to occlusion for initial studies. Distribution and expression levels of proNGF, caspase-9 and markers of edema (MMP-9 and aquaporin-4) at 4, 8, 12, 18 and 24 hours post-reperfusion will be determined by IHC and by biochemistry of brain lysates and isolated blood vessels. Neuronal processes and cell bodies will also be quantified to identify neurons and processes protected by this intervention. The cohort of animals used for the 24 hr measures will be followed over time by MRI to image infarct/edema. Edema will also be measured by EBA and by H&E staining in tissue sections.

### 6.7 Use of Anti-proNGF Antibodies to Abrogate Activation of Caspase-9

ProNGF antibodies can block the actions of proNGF without blocking the effects of mature NGF. Direct infusion of anti-proNGF into the hippocampus has been used to prevent proNGF signaling in a rodent seizure model. Le et al., The Journal of neuroscience: the official journal of the Society for Neuroscience 32 (2), 703-712 (2012). The instant experiment will illustrate the ability of anti-proNGF to inhibit proNGF to block the activation of caspase-9 and formation of edema in a stroke model. Anti-proNGF or IgG will be delivered to the mouse striatum by CED prior to MCA occlusion. Animals will be sacrificed at 1 h, 4 h, 12 h and 24 h post-reperfusion. The comparison groups will be tMCAo with anti-proNGF and tMCAo with vehicle. IHC is used to determine levels of caspase-9 in small blood vessels. To assay edema, MRI, H&E and EBA are employed. Levels of edema markers (MMP-9, Aquaporin-4) and BBB (SMI-71) will be determined biochemically. Upon inhibition of caspase-9 activity in blood vessels, proNGF will be injected during reperfusion at 4 hrs, 8 hrs or 12 hrs post-reperfusion and edema measured at 24 and 48 hours to determine at what time point post-reperfusion edema can still be inhibited. Edema will be followed by MRI, H&E and EBA.

### 6.8 Inhibition of proNGF Signaling/Level to Provide Neuroprotection

Inhibition of proNGF signaling or decreasing proNGF levels should block caspase-9 activation in blood vessels, but might not alter caspase-9 activation in neurons. Anti-proNGF or MMP-7, which will be delivered by CED prior to induction of tMCAo in rats, will be employed in these studies. Rats will be examined over 3 weeks for neurologic function and imaged by MRI for progression of edema/infarct. After sacrifice infarct volume will be determined by H&E staining. For these studies groups of 10 rats per group will be used.

### 6.9 Signaling Via p75NTR Activates Caspase-9 and Leads to Edema

p75NTR activates caspase-9 in neurons. Troy et al., J Biol Chem 277, 34295-34302 (2002). The literature and the studies described herein (FIG. 7) show that pericytes and endothelial cells in small blood vessels express p75NTR, and that tMCAo increases caspase-9 expression in these blood vessels (FIG. 4). These findings support the position that the induction of caspase-9 activity is mediated by p75NTR.

Previous studies have shown that p75NTR can increase in models of cerebral ischemia, but levels in small BVs were not studied. The expression and distribution of p75NTR will be determined during stroke by IHC of sections from the time course described above. Sections will be immunostained for p75NTR and markers of pericytes, endothelial cells, neurons, astrocytes and microglia. Pericytes/endothelial cells will be isolated and western blotting used to determine p75NTR levels at the time points used described above.

### 6.10 p75NTR Conditional Knockout Animals & Ischemia-Induced Edema

Use of p75NTR conditional knockouts mice will avoid confounding developmental abnormalities that occur in constitutive p75NTR KO mice. To specifically delete p75NTR in the endothelial cells, p75NTR floxed mice will be crossed with transgenic mice expressing Cre recombinase under the control of the Tie2 endothelial-specific promoter (Koni et al., The Journal of experimental medicine 193 (6), 741-754 (2001)). p75NTR conditional endothelial cell KO mice and wild-type littermates will be subjected to tMCAo. Animals will be examined for edema with MRI, H&E and EBA at 24 hrs post-reperfusion. Caspase-9 activation in blood vessels and striatal and dorsal cortex neurons will be measured using bVAD to capture active caspase-9. Levels of proNGF will be measured by western blotting of lysates of striatum, dorsal cortex and isolated blood vessels. IHC will be used to determine the distribution of caspase-9 and proNGF, and activity of MMP-7 and MMP-9 will be measured in sections using fluorescent zymography.

### 6.11 Use of Anti-p75NTR Antibodies to Prevent Caspase-9 Activation

p75NTR blocking antibodies block ligand binding and prevent activation of p75NTR signaling. Weskamp et al., Neuron 6 (4), 649-663 (1991). Anti-p75NTR or IgG is administered to mice using CED prior to stroke and caspase-9 activation and edema are measured at 24 hours as described above. Cultures of endothelial cells and pericytes, and of the RBE endothelial cell line will be treated with proNGF and anti-p75NTR and caspase-9 activity, ZO-1 staining and TER measured as outlined above.

### 6.12 Inhibition of p75NTR Signaling to Provide Neuroprotection

p75NTR conditional endothelial cell KO mice and wild-type littermates will be subjected to tMCAo. Mice will be examined by neurologic exams and MRI up to 1 week, 10 mice per group will be used. The p75NTR antibody studies will be performed in rats so that functional exams can be conducted for 3 weeks post-ischemia. Antibody administration will be by CED prior to the onset of occlusion. 10 rats per group. Rats will be followed by neurologic exam and MRI over 3 wks. Stroke volume will be determined by H&E after sacrifice.

### 6.13 Activated Caspase-9 Leads to Loss of Vascular Integrity

The studies described herein show that it is the specific inhibition of caspase-9 that abrogates edema in stroke. Evidence is not observed of activation of the caspase effectors, caspases-3 and -6 in the BVs. Nor do the cells with caspase-9 appear to be dying, based on nuclear morphology. These findings support the position that the function of caspase-9 in the blood vessels is the cleavage of proteins that leads to the breakdown of connections between the cells in the blood vessels. The studies described herein show that the caspase-9 inhibitor Pen1-XBIR3 attenuates expression of MMP-9 during tMCAo, supporting a function for caspase-9 upstream of the induction of MMP-9. TIMP-1 regulates MMP-9 activity, TIMP-1 contains a potential caspase-9 cleavage site (Table 1) thus caspase-9 cleavage of TIMP-1 would inhibit TIMP-1, releasing MMP-9 inhibition; MMP-9 can then degrade components of the tight junction such as claudin-5. These data support the molecular mechanisms identified herein by which caspase-9 regulates edema (e.g., FIG. 11). Other targets of caspase-9 will be assayed since other components of blood vessel integrity also have caspase-9 cleavage sites. For example, ZO-1, a component of the tight junctions, contains a caspase cleavage site, and levels of full-length ZO-1 are decreased in stroke.

bVAD has been used to capture active caspases during stroke and caspase-9 has been identified as the proximal initiator caspase activated in neurons. As shown herein, at 24 hr post-reperfusion, caspase-9 is present in BVs in stroked animals (FIG. 4). bVAD capture of caspase-9 will be used to determine the time course of caspase-9 activation in blood vessels during cerebral ischemia. Since bVAD is an inhibitor as well as an affinity ligand of caspase-9 bVAD will be administered at different times during reperfusion, to capture caspase-9 active at different time points. Mice will be treated with bVAD at different times during ischemia-reperfusion. bVAD will be given prior to occlusion, at reperfusion, at 4 hpr, 8 hpr, 12 hpr and 24 hpr. Animals will be harvested 2 hours after administration of bVAD and blood vessel cells isolated from the area of the infarct and caspase-9 activity determined by streptavidin pull-out of bVAD-caspase followed by western blot for caspase-9.

### 6.14 Caspase-9 Cleavage-Resistant TIMP-1 Protects from Edema

TIMP-1 will be engineered with a mutation of the caspase-9 cleavage site, using site directed mutatgenesis. The mutant protein will be expressed in E. coli, purified and treated with recombinant active caspase-9 and cleavage determined by western blotting. His-tagged caspase-9 cleavage resistant TIMP-1 will be expressed in pcDNA vectors and transfected into cultured RBE endothelial cells. Cultures will be treated with proNGF and cleavage of TIMP-1 assessed by western blotting. His-tags will provide identification of transfected cleavage-resistant proteins. Sister cultures will be treated with proNGF and TER measured.

### 6.15 Identification of Caspase-9 Substrates in Isolated Blood Vessels

To identify caspase-9 substrates in BVs, primary pericyte and endothelial cell co-cultures, and cultures of RBE cells will be used. Cultures will be treated with proNGF or 4-hydroxynonenal to activate caspase-9, and levels and cleavage of tight junction proteins with potential caspase-9 cleavage sites (Table 1) will be analyzed by western blotting at 4, 8, 12 and 24 hr. To determine if cleavage of substrates is mediated by caspase-9 cultures will be treated with proNGF and Pen1-XBIR3 and cleavage of tight junction proteins analyzed by western blot. Sister cultures will be treated in the same way and TER will be measured.

### 6.16 Caspase-9 Cleavage-Resistant Forms of Caspase-9 Substrates Protect from Edema in Stroke

Tight junction proteins that function as caspase-9 substrates will be engineered with mutations of the caspase-9 sites, using site directed mutagenesis. To show that the mutations block cleavage by caspase-9, the proteins will be expressed in vitro in E. coli, purified and treated with recombinant active caspase-9 and analyzed for cleavage. His-tagged caspase-9 cleavage-resistant proteins will be expressed in pcDNA vectors and transfected into cultured RBE endothelial cells. Cultures will be treated with proNGF and cleavage assessed by western blotting. His-tags will provide identification of transfected cleavage-resistant proteins. Sister cultures will be treated with proNGF and TER measured.

### 6.17 Caspase-9 Activation in MMP-9 Null Animals

MMP-9 null animals are available from Jackson Labs. The model described herein predicts that caspase-9 should be activated by tMCAo in these animals but that edema should be decreased in the animals. Caspase-9 activation in blood vessels will be determined from MMP-9 null and wild-type littermates as described above, at 0, 1, 4, 8, 12 and 24 hpr to show maximal caspase-9 activation. The development of edema will also be studied in the MMP-9 null mice utilizing MRI and EBA.

### 6.18 Materials and Methods

Convection Enhanced Delivery (CED) of Biotin-VAD-Fmk or Pen1-XBIR3.

Adult male Wistar rats (250-300 g) were anesthetized using isoflurane (2%) delivered via an anesthesia mask for stereotactic instruments (Stoelting) and positioned in a stereotactic frame. CED was performed as previously described with the following stereotactic coordinates (1 mm anterior, 3 mm lateral, 5 mm depth). (Bruce, et al., Neurosurgery 46 (3), 683-691 (2000)). Infusion of the therapeutic was then instituted at a rate of 0.5 μl/minute. Following infusion, the cannula was removed at a rate of 1 mm/minute, the burrhole was sealed with bonewax, and the skin incision was closed with skin adhesive. Postprocedure, rats were placed in a 37° C. post-operation incubator and maintained at normothermia for an hour.

Pen1-XBIR3.

The BIR3 domain from XIAP (XBIR3) was purified as previously described. (Sun, et al., J Biol Chem 275 (43), 33777-33781 (2000)). Penetratin1 (Pen1, Q-Biogene, Carlsbad, Calif.) was mixed at an equimolar ratio with purified XBIR3 and incubated overnight at 37° C. to generate disulfide-linked Pen/BIR3. Linkage was assessed by 20% SDS-PAGE and western blotting with anti-His antibody. 30 μl of Pen1-XBIR3 (36.804) was infused by ICC immediately prior to induction of ischemia. Animals were housed at room temperature, euthananized, and brains processed for immunohistochemistry (see below) or protein isolation (brain tissue dissection followed by snap-freezing in liquid nitrogen). An equivalent volume of saline was infused as a negative control.

Measures of Edema: H & E Staining.

The method published in Akpan et al., The Journal of Neuroscience 31 (24), 8894-8904 (2011) was used for H & E staining Brain sections are prepared for immunohistochemistry and stained using a hematoxylin and eosin kits from American MasterTech. Infarcted brain is visualized as an area of hematoxylin negative (pink) tissue in a surrounding background of viable (blue and pink) tissue. Serial sections are photographed and projected on tracing paper at a uniform magnification. Infarct volumes are calculated from serial sections and expressed as the percentage of infarct in the ipsilateral hemisphere and compared to percentage of infarct in the contralateral hemisphere.

Evans Blue-Albumin (EBA)

This approach is well developed for studies of pulmonary edema by J. Bhattacharya. Briefly, EBA is injected intravenously. After 30 min to 2 hours or longer, including up to and after 4 hpr, EBA concentration is assayed by spectrophotometry in samples of brain tissue obtained from different sites. Increase in EBA concentration denotes increased protein extravasation, providing regional quantification of edema formation.

Transendothelial Electrical Resistance (TER).

As described in Quadri et al., Am J Physiol Lung Cell Mol Physiol 292 (1), L334-342 (2007). For endothelial cell barrier quantification, transendothelial electrical resistance (TER) can be determined in endothelial cell monolayers grown on sterile polycarbonate inserts held at 37° C. (Endohm; World Precision Instruments, Sarasota, Fla.). After a 30-min baseline period, experimental solutions are added, and TER data were acquired every 5 sec (MP100A-CE data acquisition system, World Precision Instruments). Reported TER data are subtracted for insert resistance.

MRI.

MRI studies will be performed on a clinical GE Signa 3T HDX hardware configuration, VH3/M15 software configuration; GE Healthcare, Waukesha, Wis., USA) using a standard 8-channel human head coil as a receiver and the integrated circularly polarized body coil as the transmitter. A home made birdcage transmit/receive coil is built for rodent brain imaging with a diameter of 5 cm. Image acquisition is carried out within 5 min after administration of anesthesia. Multiple techniques or MRI modalities will be used to study stroke model. This approach will ensure that a comprehensive set of imaging evidence will be gathered. As presented here, anatomical sequences (T1W and T2W) will identify structural modification of the brain tissues while diffusion weighted images (DWI), susceptibility weighted images (SWI) and perfusion weighted images (PWI) will probe functional and physiological damages to specific regions of the brain. The high resolution anatomical images will have a resolution of 125 nanoliter voxels and low resolution functional scans will have 0.1 microliter voxels.

Blood Vessel Isolation.

Isolations will be performed as described in Quadri et al., The Journal of Biological Chemistry 278 (15), 13342-13349 (2003). Briefly, after sacrifice, brains will have meninges, cerebellum, brain stem and large blood vessels removed and ipsilateral and contralateral hemispheres will be separated. Tissue will be homogenized in DMEM and vessels isolated by differential centrifugation as described. Katyshev et al., Methods in Molecular Biology 814, 467-481 (2012). For primary cultures of endothelial cells and pericytes, blood vessels will be digested with collagenase, and cells isolated with FACS sorting. Katyshev et al., Methods in Molecular Biology 814, 467-481 (2012) and Quadri et al., The Journal of Biological Chemistry 278 (15), 13342-13349 (2003).

Endothelial Cell Line Culture.

RBE cells from ATCC will be cultured in DMEM supplemented with 5% fetal calf serum. At 10 days in culture, the presence of established tight junctions will be measured by TER (Brown et al., Brain research 1130 (1), 17-30 (2007).

In Vitro Vascular Permeability Assay.

RBE cells will be cultured on 96 well assay plates (Millipore). At 2-3 days of culture cells establish an impermeable monolayer, assessed by treatment of wells with FITC-dextran, flow-through is measured with a 96 well plate reader. The role of caspase-9 will be assessed by treating the cultures with Pen1-XBir3 with and without proNGF, and measuring permeability after 1 hr.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

What is claimed:

1. A method of treating edema comprising administering an effective amount of a caspase-9 inhibitor to a subject in need thereof.

2. The method of claim 1, wherein the caspase-9 inhibitor is administered intranasally.

3. The method of claim 2, wherein the caspase-9 inhibitor is conjugated to a cell-penetrating peptide.

4. The method of claim 3, wherein the cell-penetrating peptide is selected from the group consisting of Penetratin1, transportan, pIS1, Tat(48-60), pVEC, MAP, and MTS.

5. The method of claim 1, wherein the edema is: associated with ischemic injury in the central nervous system, edema associated with traumatic brain injury; pulmonary edema; angioedema; cardiac edema; macular edema; or peripheral edema.

6. A method according to any of the preceding claims, wherein the caspase-9 inhibitor is selected from the group consisting of: a small molecule inhibitor; a polypeptide inhibitor; and a nucleic acid inhibitor.

7. The method of claim 6, wherein the caspase-9 inhibitor is XBIR3.

8. The method of claim 7, wherein the caspase-9 inhibitor is XBIR3 linked to Penetratin1.

9. The method of claim 8, wherein the XBIR3 is liked to Penetratin1 via disulfide linkage, thereby generating a polypeptide having the amino acid sequence:
(SEQ ID NOS 2 and 11, respectively)
RQIKIWFQNRRMKWKK-s-s-NTLPRNPSMADYEARIFTFGTWIYSV
NKEQLARAGFYALGEGDKVKCFHCGGGLTDWRPSEDPWEQHARWYPG
CRYLLEQRGQEYINNIHLTHS.

10. The method of claim 1, where in the caspase-9 inhibitor is administered to a patient as a single dose or in multiple doses.

11. The method of claim 10, wherein multiple doses are administered and they are administered at intervals of: 6 times per 24 hours; 4 times per 24 hours; 3 times per 24 hours; or 2 times per 24 hours.

12. The method of claim 10, wherein the dose or doses administered comprise the caspase-9 inhibitor at a concentration of: 0.01 μM to 1000 μM; 1 μM to 500 μM; or 10 μM to 100 μM.


SEQUENCE LISTING (12 sequences)

SEQ ID NO: 1 - protein, 4 aa, Artificial Sequence
  Description of Artificial Sequence Synthetic peptide
VEID

SEQ ID NO: 2 - protein, 16 aa, Drosophila sp.
RQIKIWFQNRRMKWKK

SEQ ID NO: 3 - protein, 16 aa, Artificial Sequence
  Description of Artificial Sequence Synthetic peptide
RRLRRLLRRLLRRLRR

SEQ ID NO: 4 - protein, 41 aa, Artificial Sequence
  Description of Artificial Sequence Synthetic polypeptide
YTIWMPENPRPGTPCDIFTNSRGKRASNGGGGRRRRRRRRR

SEQ ID NO: 5 - protein, 27 aa, Artificial Sequence
  Description of Artificial Sequence Synthetic peptide
GWTLNSAGYLLGKINLKALAALAKKIL

SEQ ID NO: 6 - protein, 16 aa, Artificial Sequence
  Description of Artificial Sequence Synthetic peptide
PVIRVWFQNKRCKDKK

SEQ ID NO: 7 - protein, 13 aa, Human immunodeficiency virus
GRKKRRQRRRPPQ

SEQ ID NO: 8 - protein, 16 aa, Artificial Sequence
  Description of Artificial Sequence Synthetic peptide
LLIILRRRIRKQAHAH

SEQ ID NO: 9 - protein, 27 aa, Artificial Sequence
  Description of Artificial Sequence Synthetic peptide
GALFLGWLGAAGSTMGAWSQPKKKRKV

SEQ ID NO: 10 - protein, 18 aa, Artificial Sequence
  Description of Artificial Sequence Synthetic peptide
KLALKLALKALKAALKLA

SEQ ID NO: 11 - protein, 94 aa, Artificial Sequence
  Description of Artificial Sequence Synthetic polypeptide
NTLPRNPSMADYEARIFTFGTWIYSVNKEQLARAGFYALGEGDKVKCFHCGGGLTDWRPSEDPWEQHARWYPGCRYLLEQRGQEYINNIHLTHS

SEQ ID NO: 12 - protein, 4 aa, Artificial Sequence
  Description of Artificial Sequence Synthetic peptide
DEVD